(19) United States
(12) Patent Application Publication
(10) Pub. No.: US 2014/0187995 A1
(43) Pub. Date: Jul. 3, 2014

(54) ELECTRODE PATCH MODULE AND CHRONIC PAIN DETECTION METHOD AND APPARATUS THEREOF

(71) Applicant: INDUSTRIAL TECHNOLOGY RESEARCH INSTITUTE, Hsinchu (TW)

(72) Inventors: Wei-Wen Hu, Kaohsiung City (TW); Yung-Ching Huang, Taipei City (TW); Yu-Wei Hung, Tainan City (TW)

(73) Assignee: INDUSTRIAL TECHNOLOGY RESEARCH INSTITUTE, Hsinchu (TW)

(21) Appl. No.: 13/775,264

(22) Filed: Feb. 25, 2013

(30) Foreign Application Priority Data
Jan. 2, 2013 (TW) 102100015

(51) Int. Cl. A61B 5/0492

(52) U.S. Cl. CPC A61B 5/0492
USPC 600/546

(57) ABSTRACT

An electrode patch module, a chronic pain detection method and an apparatus thereof are provided. The electrode patch module affixed at a zone to be tested contains at least three electrode pads. The chronic pain detection apparatus selects multiple electrode pairs from the electrode pads and uses the electrode pairs to measure the zone to be tested so as to obtain multiple characteristic values of different muscle areas. The chronic pain detection apparatus receives the characteristic values for performing comparison operations to produce a result for assistant judging where the chronic pain may have in the zone to be tested.

### CROSS-REFERENCE TO RELATED APPLICATION

This application claims the priority benefit of Taiwan application serial no. 102100015, filed on Jan. 2, 2013. The entirety of the above-mentioned patent application is hereby incorporated by reference herein and made a part of this specification.

### TECHNICAL FIELD

The disclosure relates to an electrode patch module and a chronic pain detection method and an apparatus thereof.

### BACKGROUND

The physiological signal apparatus is used to measure physiological signals through placing/affixing electrode patches on the human. The conventional electrode patch has only two electrodes, i.e., a positive electrode and a negative electrode, respectively. Usually, the positive electrode and the negative electrode need to be correctly placed/affixed on a same fascicle of a human body to collect micro-physiological signals. However, at different portions of the human body, muscle stria directions (the fascicle directions) are different from each other. In order to measure correct physiological signals through the electrode patches, professionals familiar with the muscle stria directions are required, so that the positive electrode and the negative electrode are correctly placed/affixed on the same fascicle. Otherwise, it is unable to properly use the traditional electrode patches and obtain the correct physiological signals.

### SUMMARY

An embodiment of the disclosure provides a chronic pain detection method by using electrode patch module. The chronic pain detection method includes: providing an electrode patch module, in which the electrode patch module contains at least three electrode pads and a plurality of electrode pairs are selected from the electrode pads; measuring electromyography signals (EMG signals) of the electrode pairs to obtain at least one characteristic value; and performing a comparison operation on the characteristic values to produce a judgment result.

An embodiment of the disclosure provides a chronic pain detection apparatus, which includes an electrode patch module, a signal-capturing module and a control unit. The electrode patch module contains at least three electrode pads. The signal-capturing module is coupled to the electrode patch module, in which the signal-capturing module selects a plurality of electrode pairs in the electrode pads. The control unit is coupled to the signal-capturing module, the control unit measures EMG signals of the electrode pairs through the signal-capturing module to obtain at least one characteristic value. The control unit performs a comparison operation on the characteristic values to produce a judgment result.

An embodiment of the disclosure provides an electrode patch module, which contains at least three electrode pads. The electrode pads form a plurality of electrode pairs, the electrode pairs respectively have a first electrode pad and a second electrode pad, the first electrode pad and the second electrode pad in a same electrode pair have an interval therebetween, and the intervals of the electrode pairs are equal to each other.

Several exemplary embodiments accompanied with figures are described in detail below to further describe the disclosure in details.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 is a schematic function block diagram of a chronic pain detection apparatus according to an embodiment of the disclosure.

FIG. 2 is a schematic circuit block diagram illustrating a signal-capturing module synchronously measures different electrode pairs according to an embodiment of the disclosure.

FIG. 3 is a schematic circuit block diagram illustrating a signal-capturing module asynchronously measures different electrode pairs according to another embodiment of the disclosure.

FIG. 4 is a flowchart illustrating a chronic pain detection method by using an electrode patch module according to an embodiment of the disclosure.

FIG. 5 is a flowchart illustrating a chronic pain detection method by using an electrode patch module according to another embodiment of the disclosure.

FIG. 6 is a schematic layout diagram of the electrode patch module of FIG. 1 according to an embodiment of the disclosure.

FIG. 7 is a schematic layout diagram of the electrode patch module of FIG. 1 according to another embodiment of the disclosure.

FIG. 8 is a schematic layout diagram of the electrode patch module of FIG. 1 according to yet another embodiment of the disclosure.

FIG. 9 is a schematic layout diagram of the electrode patch module of FIG. 1 according to further another embodiment of the disclosure.

FIG. 10 is a schematic layout diagram of the electrode patch module of FIG. 1 according to further another embodiment of the disclosure.

FIG. 11 is a schematic layout diagram of the electrode patch module of FIG. 1 according to further another embodiment of the disclosure.

FIG. 12 is a schematic layout diagram of the electrode patch module of FIG. 1 according to other embodiments of the disclosure.

FIG. 13 is an application situation diagram of the electrode patch module of FIG. 12 according to an embodiment of the disclosure.

### DETAILED DESCRIPTION OF DISCLOSED EMBODIMENTS

The word “coupling” used in the specification of the disclosure (including the “what is claimed is”) means any direct or indirect connection means. For example, “the first device is coupled to the second device” in the specification should be interpreted as “the first device can be directly connected to the second device” or “the first device can be indirectly connected to the second device through other devices or a sort of connection means”. In addition, wherever possible, the same reference numbers of components/parts/steps are used in the drawings and the description to refer to the same or like portions. The components/parts/steps using the same reference numerals or using the same terminology in the different embodiments can get cross-reference instructions.

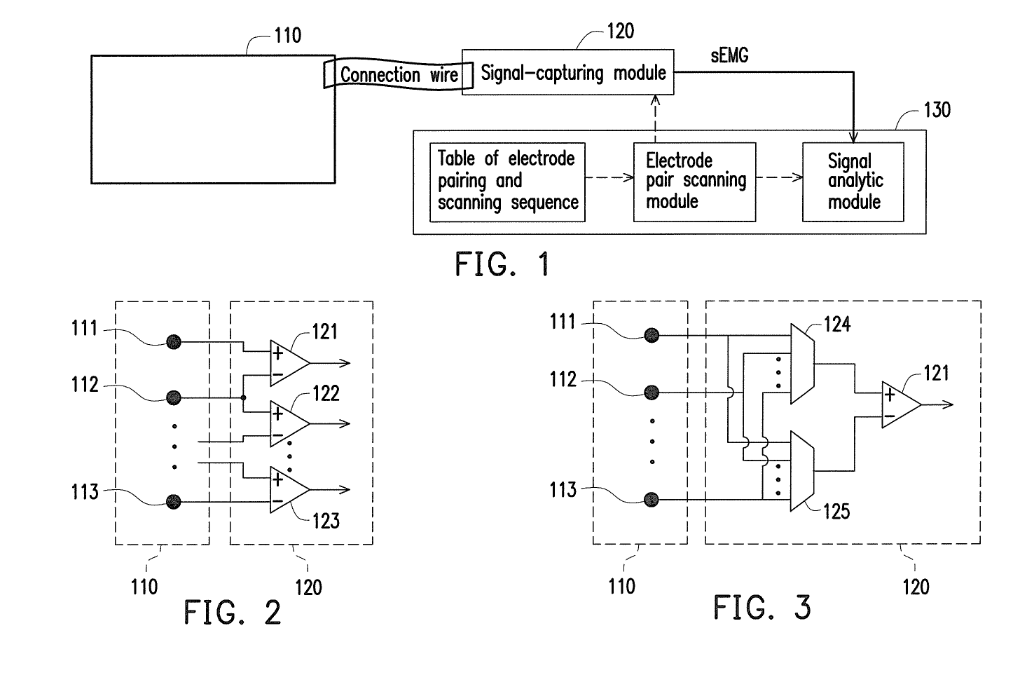

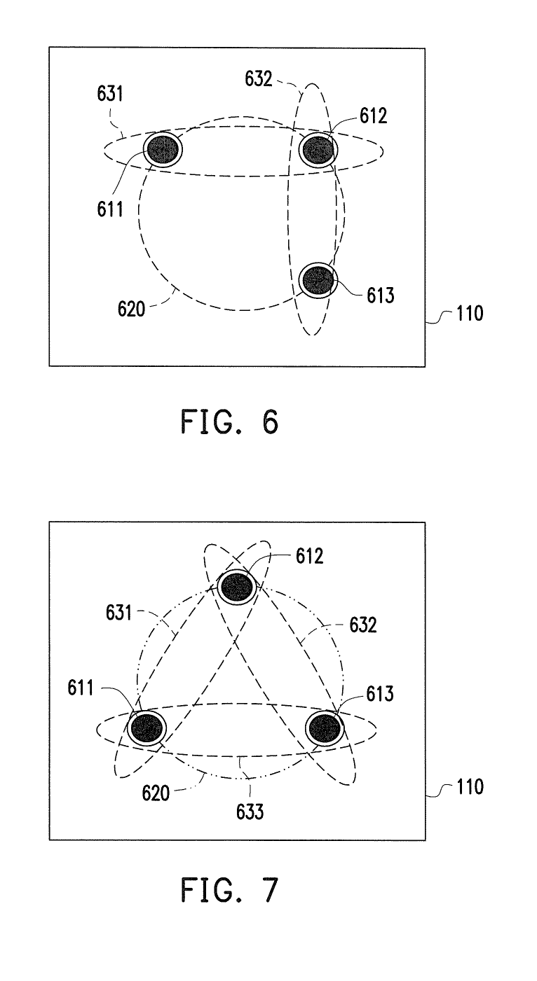

FIG. 1 is a schematic function block diagram of a chronic pain detection apparatus according to an embodiment of the disclosure. The chronic pain detection apparatus includes an electrode patch module 110, a signal-capturing module 120 and a control unit 130. The chronic pain detection apparatus can employ one or a plurality of electrode patch modules 110 according to application demands of different embodiments. The electrode patch module 110 can affix to a zone to be tested on an organism (e.g., human body) skin surface. The electrode patch module 110 contains at least three electrode pads. The quantity and geometric position of the electrode pads in the electrode patch module 110 can have different layouts according to application demands of different embodiments. For example, the electrode pads in the electrode patch module 110 can be placed with certain geometric positions to meet the fascicle direction in the zone to be tested. The different embodiments of the electrode patch module 110 can later refer to the related descriptions of FIGS. 6-12 for analogy to the rests.

All the electrode pads in the electrode patch module 110 are coupled to the signal-capturing module 120 via the corresponding connection wires. The signal-capturing module 120 can select/define a plurality of electrode pairs from the electrode pads in the electrode patch module 110 according to the control of the control unit 130, in which the electrode pairs are respectively corresponding to different muscle areas in the zone to be tested. The signal-capturing module 120 can synchronously or asynchronously capture electromyography signals (EMG signals) sEMG measured by the electrode pairs on the electrode patch module 110.

For example, in some embodiments, the signal-capturing module 120 can synchronously (simultaneously) measure the EMG signals of the corresponding muscle areas via all the electrode pairs on the electrode patch module 110. FIG. 2 is a schematic circuit block diagram illustrating the signal-capturing module 120 synchronously measures different electrode pairs according to an embodiment of the disclosure. The embodiment of FIG. 2 and the embodiment of FIG. 1 can get cross-reference instructions. The signal-capturing module 120 includes a plurality of amplifiers (for example, amplifiers 121, 122 and 123), and these amplifiers can be operation amplifier, error amplifier, voltage comparator or other amplifying circuits. The electrode patch module 110 of FIG. 2 contains a plurality of electrode pads (for example, electrode pads 111, 112 and 113). It should be noted that the quantity and geometric position of the electrode pads in the electrode patch module 110 are not limited by the layout of FIG. 2.

The electrode pads 111-113 are respectively electrically connected to the corresponding amplifiers in the signal-capturing module 120 via the connection wires. For example, the electrode pad 111 is electrically connected to the non-inverting input terminal of the amplifier 121, while the electrode pad 112 is electrically connected to the inverting input terminal of the amplifier 121. Thus, the electrode pad 111 and the electrode pad 112 can be selected as an electrode pair, in which the electrode pad 111 is the positive electrode and the amplifier 122 is the negative electrode. At the time, the output terminals of the amplifier 121 can output the EMG signal of the muscle area corresponding to this electrode pair (i.e., the electrode pads 111 and 112). On the other hand, the electrode pad 112 is further electrically connected to the non-inverting input terminal of the amplifier 122. Thus, the electrode pad 112 can be selected as the positive electrode of the other electrode pair, so that the output terminals of the amplifier 122 can output the EMG signal of the muscle area corresponding to the other electrode pair.

In some embodiments, the outputs of the amplifiers 121-123 can be processed by a signal processing circuit (not shown, such as rear-stage amplification, noise filtering and/or other signal processing), followed by making the processing result as the EMG signal sEMG output from the signal-capturing module 120. In some other embodiments, the outputs of the amplifiers 121-123 can directly serve as the EMG signal sEMG output from the signal-capturing module 120.

In some other embodiments, the signal-capturing module 120 of FIG. 1 can scan all the electrode pairs on the electrode patch module 110 one by one so as to measure the EMG signals of the corresponding muscle areas. FIG. 3 is a schematic circuit block diagram illustrating the signal-capturing module 120 asynchronously measures different electrode pairs according to another embodiment of the disclosure. The embodiment of FIG. 3 can refer to the descriptions of the embodiments in FIGS. 1 and 2 for analogy to the rests. The signal-capturing module 120 includes an amplifier 121 and two multiplexers 124 and 125.

The electrode pads 111-113 on the electrode patch module 110 are respectively electrically connected to the multiplexers 124 and 125 in the signal-capturing module 120 via the connection wires. The multiplexer 124 can, according to the control of the control unit 130, select one from the electrode pads 111-113 to be electrically connected to the non-inverting input terminal of the amplifier 121, and the multiplexer 125 can select another one from the electrode pads 111-113 to be electrically connected to the inverting input terminal of the amplifier 121. For example, when the multiplexer 124 and the multiplexer 125 respectively select the electrode pad 111 and the electrode pad 112 (i.e., the electrode pad 111 and the electrode pad 112 are selected as the positive electrode and the negative electrode of an electrode pair), the output terminals of the amplifier 121 can output the EMG signals of the muscle area corresponding to the electrode pair (i.e., the electrode pads 111 and 112). Analogy to the rests, the multiplexers 124 and 125 can select a plurality of electrode pairs in all the electrode pads on the electrode patch module 110 according to the control of the control unit 130. By controlling the multiplexers 124 and 125, the amplifier 121 can scan all the electrode pairs on the electrode patch module 110 one by one so as to measure the EMG signals of the corresponding muscle areas.

In some embodiments, the output of the amplifier 121 can be processed by a signal processing circuit (not shown, such as rear-stage amplification, noise filtering and/or other signal processing), followed by making the processing result as the EMG signal sEMG output from the signal-capturing module 120. In some other embodiments, the output of the amplifier 121 can directly serve as the EMG signal sEMG output from the signal-capturing module 120.

Referring to FIG. 1 the signal-capturing module 120 is coupled to the control unit 130. The control unit 130, through the signal-capturing module 120 and the electrode pairs on the electrode patch module 110, measures the zone to be tested so as to obtain characteristic values of different muscle areas in the zone to be tested. The control unit 130 can perform a comparison operation on the characteristic values to produce a judgment result. In some embodiments, the judgment result contains judging whether chronic pain is present at these muscle areas.

According to application demands of different embodiments, the control unit 130 can be a single chip (for example, microprocessor chip or other chips) or a circuit comprised by a plurality of components/chips. In the embodiment, the control unit 130 contains a table of electrode pairing and scanning sequence, an electrode pair scanning module and a signal analytic module. The table of electrode pairing and scanning sequence contains one or multiple lookup tables to facilitate record the pairing relationship between the electrode pads on the electrode patch module 110. In some embodiments, when the signal-capturing module 120 measures in scanning way all the electrode pairs on the electrode patch module 110, the sequence for the signal-capturing module 120 to scan the electrode pairs on the electrode patch module 110 can be also recorded in the table of electrode pairing and scanning sequence.

The electrode pair scanning module is coupled to the signal-capturing module 120 and the signal analytic module. The electrode pair scanning module can access the pairing relationship data recorded in the table of electrode pairing and scanning sequence for controlling the signal-capturing module 120 according to the pairing relationship. As per the control of the electrode pair scanning module, the signal-capturing module 120 can select/define a plurality of electrode pairs from the electrode pads in the electrode patch module 110. The electrode pair scanning module can also access the sequence data recorded in the table of electrode pairing and scanning sequence so as to control the signal-capturing module 120 according to the sequence. As per the control of the electrode pair scanning module, the signal-capturing module 120 can scan all the electrode pairs in the electrode patch module 110 according to a predetermined sequence.

The signal analytic module is coupled to the signal-capturing module 120. By using the signal analytic module, the EMG signal sEMG output from the electrode pair scanning module can be analyzed. During scanning the electrode pairs in the electrode patch module 110 by the signal-capturing module 120 under the control of the electrode pair scanning module, the electrode pair scanning module can synchronously inform the signal analytic module of the present scanning state. Therefore, the signal analytic module is aware of the EMG signal sEMG output presently from the signal-capturing module 120 belongs to which electrode pair in the electrode patch module 110.

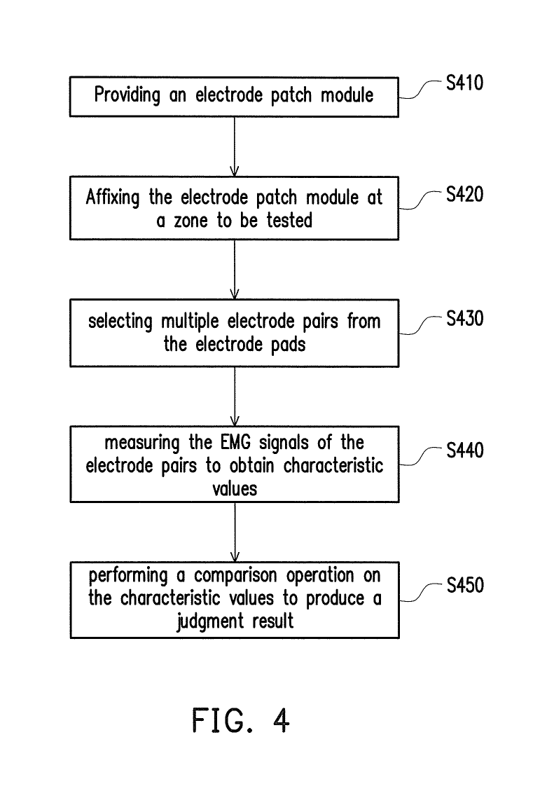

FIG. 4 is a flowchart illustrating a chronic pain detection method by using an electrode patch module according to an embodiment of the disclosure. The embodiment of FIG. 4 can refer to the descriptions of the embodiments in FIGS. 1-3 for analogy to the rests. Referring to FIGS. 1-4, first in step S410, one or multiple electrode patch modules 110 are provided, in which the electrode patch module 110 contains at least three electrode pads.

Next in step S420, the electrode patch module 110 is affixed at the zone to be tested on organism (e.g., human body) skin surface. In step S430, the signal-capturing module 120 selects a plurality of electrode pairs from the electrode pads according to the control of the control unit 130. In the embodiment, the electrode pairs are respectively corresponding to different muscle areas of the zone to be tested. In step S440, the control unit 130 measures the EMG signals of the electrode pairs to obtain at least one characteristic value. In step S450, the control unit 130 performs a comparison operation on the characteristic values to produce a judgement result. In some embodiments, the judgement result contains judging whether chronic pain is present at different muscle areas. The judgement result herein can aid professionals for auxiliary reference during diagnoses.

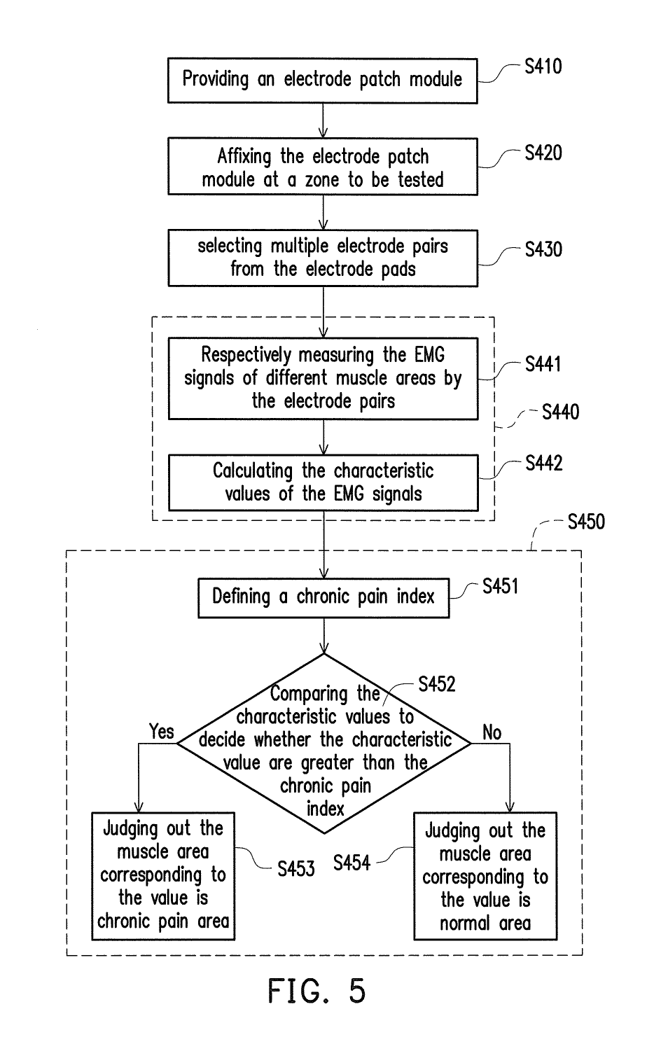

FIG. 5 is a flowchart illustrating a chronic pain detection method by using an electrode patch module according to another embodiment of the disclosure. The embodiment of FIG. 5 can refer to the descriptions of the embodiments in FIGS. 1-4 for analogy to the rests. The difference of the embodiment from the embodiment of FIG. 4 rests in that step S440 in the embodiment of FIG. 5 contains step S441 and step S442, and step S450 contains step S451, step S452 and step S453.

Referring to FIG. 1 and FIG. 5, the signal-capturing module 120 performs step S441 for respectively measuring the EMG signals sEMG of different muscle areas of the zone to be tested through the electrode pairs selected in step S430 and sending the EMG signals sEMG of the muscle areas to the control unit 130. The control unit 130 performs step S442 to respectively calculate the characteristic values of the EMG signals sEMG of the muscle areas. In step S442, the characteristic values are calculated with any algorithms. For example, the control unit 130 can respectively calculate the average amplitudes of the EMG signals sEMG of the muscle areas, followed by respectively taking the average amplitudes by the control unit 130 as the characteristic values of the muscle areas. For example, in FIG. 2, the control unit 130 can measure the EMG signals sEMG of the corresponding muscle areas (referred as target areas herein) for many times in the predetermined duration through the amplifier 121 and the electrode pads 111 and 112 so as to obtain a plurality of electromyography values. Then, the control unit 130 calculates out the average amplitudes of the EMG signals sEMG of the target areas in the predetermined duration according to the electromyography values, followed by taking the average amplitudes as the characteristic values of the target areas.

The comparison operation in step S450 can be performed with any comparison algorithm. For example, steps S451-S454 in FIG. 5 describe only one of implementation examples of the comparison operation. Step S451 defines a chronic pain index. In some embodiments, the chronic pain index can be a constant value determined according to the design requirement of a real system. In some other embodiments, the chronic pain index can be a varied parameter value adjusted according to the user requirement. In the rest embodiments, step S410 can include: comparing the characteristic values of all (or partial) muscle areas in the zone to be tested with each other by the control unit 130 so as to find out a minimal value among the characteristic values; and setting N times of the minimal value as the chronic pain index by the control unit 130 (N is a positive real number). The value N can be determined according to the design requirement of a real system or adjusted according to the user requirement.

According to the disclosure, it is revealed that for a patient with long-term lower-back chronic pain, the average amplitude of the EMG signals sEMG under a static test tends to be larger in comparison with a normal person. According to the experimental statistics results, the average amplitude of the EMG signals sEMG for a subject with long-term lower-back chronic pain under a static test, the average amplitude of the EMG signals sEMG under a static test is greater than the average amplitude of a healthy subject (over three times). Thus, in the embodiment, the chronic pain index of step S451 is set with three times of the minimal value (the minimal value among the characteristic values of all the muscle areas in the zone to be tested).

The control unit 130 performs step S452 to decide whether the characteristic value of every muscle area is greater than the chronic pain index defined by step S451 through comparison operations. If one of the characteristic values is greater than or equal to the chronic pain index, the control unit 130 judges out the muscle area corresponding to the value is chronic pain area (step S453); otherwise, if one of the characteristic values is less than the chronic pain index, the control unit 130 judges out the muscle area corresponding to the value is normal area (step S454). The above-mentioned judgment result can aid professionals for diagnose reference.

In summary, the disclosure uses a plurality of electrode pairs for measuring the characteristic values (e.g., EMG signals) of a zone to be tested and analysing the characteristic values so as to judge whether the different muscle areas corresponding to the electrode pairs are chronic pain areas.

FIG. 6 is a schematic layout diagram of the electrode patch module 110 of FIG. 1 according to an embodiment of the disclosure. The embodiment of FIG. 6 can refer to the descriptions of the embodiments in FIGS. 1-5 for analogy to the rests. In the embodiment, the electrode patch module 110 contains an electrode pad 611, an electrode pad 612 and an electrode pad 613. The electrode pads 611-613 can refer to the descriptions of the electrode pairs 111-113 in FIG. 3 or 4 to get understanding by analogy.

Referring to FIGS. 1, 4 and 6, the electrode patch module 110 has the electrode pads 611-613 and the electrode pads 611-613 are disposed on a quasi-circle 620, as shown by FIG. 6. The geometric shape of quasi-circle 620 contains circle and oval, which the disclosure is not limited to. In step S420, the user can affix the electrode patch module 110 at a consciously-felt chronic pain place by a user (i.e., a zone to be tested) without considering the fascicle direction. The electrode pads 611-613 form a plurality of electrode pairs. For example, in step S430, the signal-capturing module 120 can select the electrode pads 611 and 612 to form an electrode pair 631 and select the electrode pads 612 and 613 to form another electrode pair 632. There is an interval between a first electrode pad and a second electrode pad in a same electrode pair, and the intervals of different electrode pads are equal to each other. For example, the interval between the electrode pads 611 and 612 in the electrode pair 631 is equal to the interval between the electrode pads 612 and 613 in the electrode pair 632.

An axis is defined between the first electrode pad and the second electrode pad in a same electrode pair. In the embodiment, the axis of the electrode pair 631 is not parallel to the axis of the electrode pair 632. The control unit 130 in step S440 respectively obtains the characteristic values of the muscle areas where the electrode pair 631 and the electrode pair 632 are located at. In step S450, the control unit 130 judges whether the muscle areas where the electrode pair 631 and the electrode pair 632 are located at are chronic pain areas. If the characteristic value of only the muscle area of the electrode pair 632 among the electrode pairs 631 and 632 is greater than the chronic pain index, it indicates the included angle between the axis direction of the electrode pair 632 and the fascicle direction is very small, so that the control unit 130 can judge out the chronic pain spot is located within the muscle area where the electrode pair 632 is located at or nearby.

If the characteristic values of the muscle areas of the electrode pairs 631 and 632 are very close to each other (even, equal to each other) and both the characteristic values of the muscle areas of the electrode pairs 631 and 632 are greater than a second chronic pain index (the second chronic pain index herein is set with two times of the minimal value among the characteristic values of the muscle areas where the electrode pairs 631 and 632 are located at or other reference values), it indicates the chronic pain spot and the fascicle direction should be within the included angle between the axis of the electrode pair 631 and the axis of the electrode pair 632. Consequently, the control unit 130 can deduce the included angle between the fascicle direction and the axis direction of the electrode pair 631 (or deduce the included angle between the fascicle direction and the axis direction of the electrode pair 632) so as to judge the position of the chronic pain spot according to the ratio between the two characteristic values of the muscle areas where the electrode pair 631 and the electrode pair 632 are respectively located at.

It can be seen for a common user unfamiliar with the muscle stria directions to use the electrode patch module 110, the chronic pain detection apparatus of the disclosure still can detect out the chronic pain location in the zone to be tested through the electrode patch module 110 as shown by FIG. 6. However, the implementation of the electrode patch module 110 in FIG. 1 should not be limited by FIG. 6, referring to the following FIG. 7 as an example.

FIG. 7 is a schematic layout diagram of the electrode patch module 110 of FIG. 1 according to another embodiment of the disclosure. The embodiment of FIG. 7 can refer to the descriptions of the embodiments in FIGS. 1-6 for analogy to the rests. The difference of FIG. 7 from the embodiment of FIG. 6 rests in that in addition to the electrode pairs 631 and 632, in the embodiment of FIG. 7, the signal-capturing module 120 can further select the electrode pads 611 and 613 to form an electrode pair 633 in step S430. In the embodiment, the interval between the electrode pads 611 and 612, the interval between the electrode pads 612 and 613 and the interval between the electrode pads 613 and 611 are equal to each other. The axes of the electrode pairs 631, 632 and 633 are not parallel to each other.

The control unit 130 sequentially scan the electrode pairs 631, 632 and 633 through the signal-capturing module 120. According to the scanning result, the control unit 130 in step S450 can judge out which electrode pair is parallel to the fascicle direction (or the included angle between the axis direction of which electrode pair and the fascicle direction is minimum) so as to further judge the position of chronic pain. The operation details of the embodiment of FIG. 7 can refer to the related description of FIG. 6, which are omitted to describe.

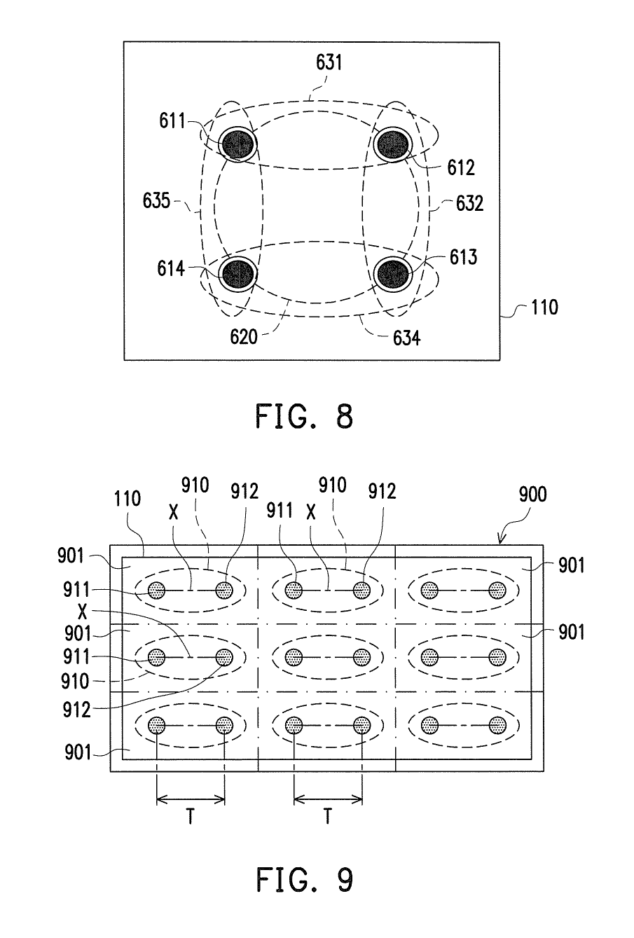

FIG. 8 is a schematic layout diagram of the electrode patch module 110 of FIG. 1 according to yet another embodiment of the disclosure. The embodiment of FIG. 8 can refer to the descriptions of the embodiments in FIGS. 1-7 for analogy to the rests. The difference of FIG. 8 from the embodiment of FIG. 6 rests in that in addition to the electrode pads 611-613, in the embodiment of FIG. 8, the electrode patch module 110 further includes an electrode pad 614. The electrode pads 611-614 are disposed on a quasi-circle 620, as shown by FIG. 8. In the embodiment, the interval between the electrode pads 611 and 612, the interval between the electrode pads 612 and 613, interval between the electrode pads 613 and 614 and the interval between the electrode pads 614 and 611 are equal to each other.

In some embodiments, the signal-capturing module 120 can select the electrode pads 611 and 612 to form an electrode pair 631 and select the electrode pads 614 and 613 to form an electrode pair 634 in step S430. The axes of the electrode pairs 631 and 634 are parallel to each other. The control unit 130 sequentially scan the electrode pairs 631 and 634 through the signal-capturing module 120. In some other embodiments, the signal-capturing module 120 in step S430 can select the electrode pads 611 and 614 to form an electrode pair 635 and select the electrode pads 612 and 613 to form an electrode pair 632. The axes of the electrode pairs 632 and 635 are parallel to each other. The control unit 130 sequentially scan the electrode pairs 635 and 632 through the signal-capturing module 120. In the rest embodiments, the signal-capturing module 120 in step S430 can select the electrode pads 611 and 612 to form an electrode pair 631, select the electrode pads 612 and 613 to form an electrode pair 632, select the electrode pads 613 and 614 to form an electrode pair 634 and select the electrode pads 614 and 611 to form an electrode pair 635. The control unit 130 sequentially scan the electrode pairs 631, 632, 634 and 635 through the signal-capturing module 120. According to the scanning result, in step S450, the control unit 130 can judge out the position of chronic pain spot. The operation details of the embodiment of FIG. 8 can refer to the related description of FIG. 6, which are omitted to describe.

FIG. 9 is a schematic layout diagram of the electrode patch module 110 of FIG. 1 according to further another embodiment of the disclosure. The embodiment of FIG. 9 can refer to the descriptions of the embodiments in FIGS. 1-6 for analogy to the rests. Referring to FIGS. 1, 5 and 9, the electrode patch module 110 contains a plurality of electrode pairs 910, in which each of the electrode pairs 910 respectively has a first electrode pad 911 and a second electrode pad 912. In a same electrode pairs 910, there is an interval T between the first electrode pad 911 and the second electrode pad 912 and an axis X is defined between the first electrode pad 911 and the second electrode pad 912, as shown by FIG. 9. The intervals T of different electrode pairs 910 are equal to each other and the axes X of different electrode pairs 910 are parallel to each other. In step S420, the electrode patch module 110 is affixed at a zone to be tested 900, and the zone to be tested 900 has a plurality of muscle areas 901, in which each of the muscle areas 901 is corresponding to a corresponding electrode pairs 910.

In step S441, the control unit 130 can measure an EMG signals sEMG on each of the electrode pairs 910 through the signal-capturing module 120. In the embodiment, the control unit 130 can synchronously measure an EMG signals sEMG on each of the electrode pairs 910 through the signal-capturing module 120. In step S442, the control unit 130 can calculate the characteristic value of the EMG signals sEMG measured by each of the electrode pairs 910. In the embodiment, the characteristic value is the average amplitude of the EMG signals. In step S451, the control unit 130 compares all the characteristic values with each other to obtain the minimal value among the characteristic values. In the embodiment, the control unit 130 makes all the characteristic values arranged in large-to-small order to obtain the minimal value among the characteristic values. In step S451, the control unit 130 further defines the chronic pain index with N times of the minimal value among the characteristic values. In the embodiment, N is equal to three. In step S452, the control unit 130 compares the characteristic values with the chronic pain index. If a characteristic value is greater than or equal to the chronic pain index, the control unit 130 judges out the state of the corresponding EMG signal is chronic pain and judges out the muscle area 901 of the corresponding electrode pair 910 is chronic pain (step S453). If a characteristic value is less than the chronic pain index, the control unit 130 judges out the state of the corresponding EMG signal is normal and further judges out the muscle area 901 corresponding to the electrode pair 910 is normal (step S454). As the description above, the judgement results are used to aid professionals for auxiliary diagnoses.

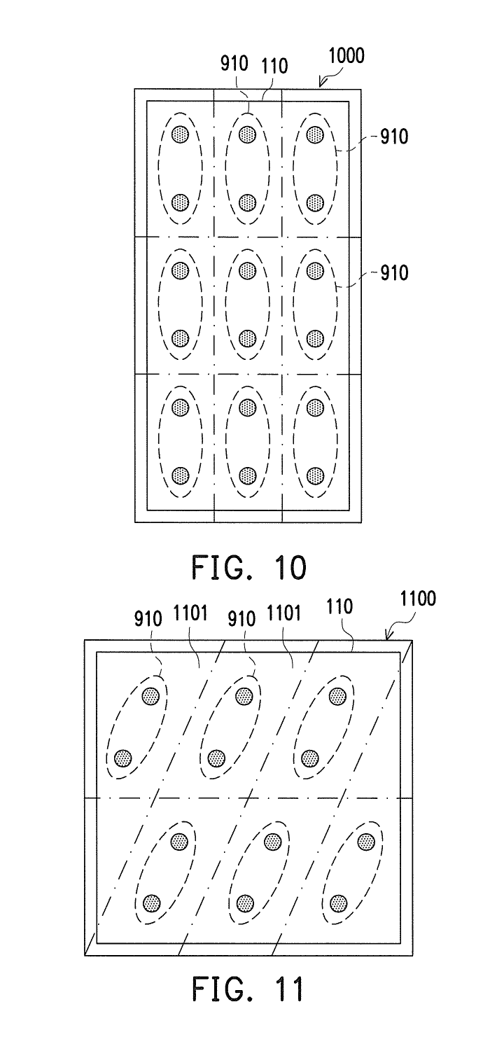

At different positions, the muscle stria directions on a human body are different. The different embodiments of the disclosure can use different arrangements of the electrode pairs to make the electrode patch module 110 suitable for muscle strias of different positions, in which the EMG signal measuring ways of the electrode patch module 110 in different arrangements are different as well. In FIG. 9, the axes X of the electrode pairs 910 of the electrode patch module 110 are in horizontal arrangement and the measuring way of the EMG signals sEMG can be synchronous measurement or in measurement orders of from top to bottom and left to right to sequentially scan and measure the electrode pairs 910. However, the implementation of the electrode patch module 110 should not be limited by the above-mentioned embodiments, referring to the following FIG. 10 as an example.

FIG. 10 is a schematic layout diagram of the electrode patch module 110 of FIG. 1 according to further another embodiment of the disclosure. The electrode patch module 110 is affixed at a zone to be tested 1000. The embodiment of FIG. 10 can refer to the descriptions of the embodiments in FIGS. 1-9 for analogy to the rests. The difference of FIG. 10 from the embodiment of FIG. 9 rests in that in the electrode patch module 110 of the embodiment of FIG. 10, the axes X of the electrode pairs 910 are in longitudinal arrangement and the measuring way of the EMG signals sEMG can be synchronous measurement or in measurement orders of from bottom to top and left to right to sequentially scan and measure the electrode pairs 910.

FIG. 11 is a schematic layout diagram of the electrode patch module 110 of FIG. 1 according to further another embodiment of the disclosure. The electrode patch module 110 is affixed at a zone to be tested 1100. The embodiment of FIG. 11 can refer to the descriptions of the embodiments in FIGS. 1-10 for analogy to the rests. The difference of FIG. 11 from the embodiment of FIG. 10 rests in that in the electrode patch module 110 of the embodiment of FIG. 11, the axes X of the electrode pairs 910 are in longitudinal oblique arrangement and the measuring way of the EMG signals sEMG can be synchronous measurement or in clockwise order to sequentially scan and measure the electrode pairs 910, or in measurement orders of from top to bottom and left to right to sequentially scan and measure the muscle area 1101 where the electrode pairs 910 are located at.

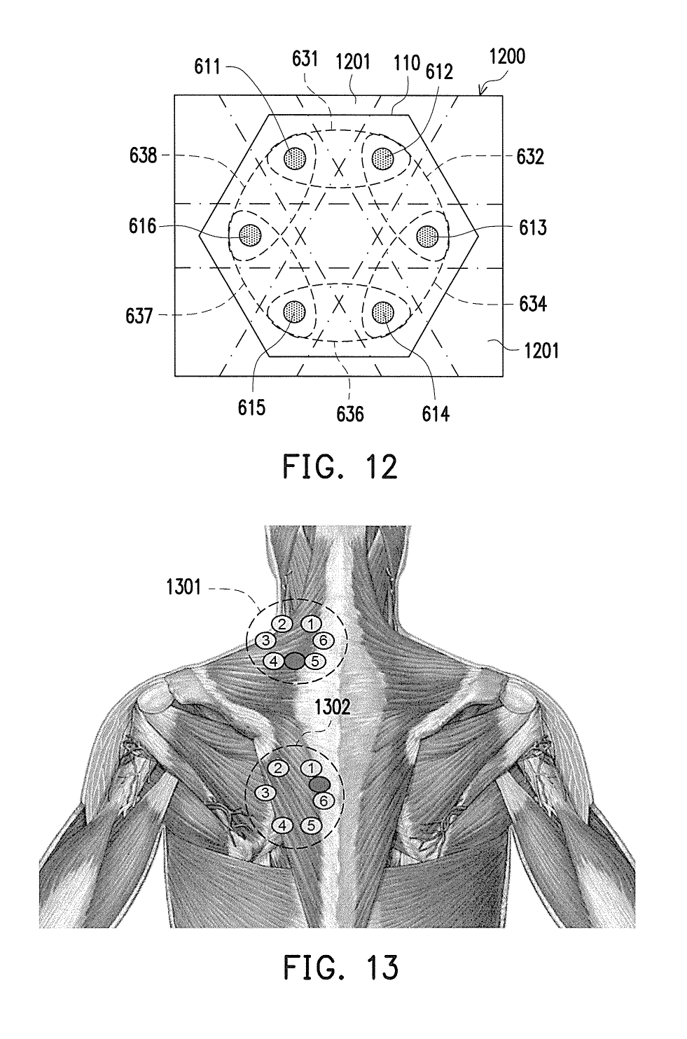

FIG. 12 is a schematic layout diagram of the electrode patch module 110 of FIG. 1 according to other embodiments of the disclosure. The electrode patch module 110 is affixed at a zone to be tested 1200. The embodiment of FIG. 12 can refer to the descriptions of the embodiments in FIGS. 1-11 for analogy to the rests. The difference of FIG. 12 from the embodiment of FIG. 7 rests in that, in addition to the electrode pads 611, 612 and 613, the electrode patch module 110 of the embodiment of FIG. 12 further includes electrode pads 614, 615 and 616. The electrode pads 611-616 are disposed on a quasi-circle 620 in surrounding arrangement, as shown by FIG. 12. In the embodiment, the interval between the electrode pads 611 and 612, the interval between the electrode pads 612 and 613, the interval between the electrode pads 613 and 614, the interval between the electrode pads 614 and 615, the interval between the electrode pads 615 and 616 and the interval between the electrode pads 616 and 611 are equal to each other.

In this embodiment, the signal-capturing module 120 can select the electrode pads 611 and 612 to form an electrode pair 631, the electrode pads 612 and 613 to form an electrode pair 632, the electrode pads 613 and 614 to form an electrode pair 634, the electrode pads 614 and 615 to form an electrode pair 636, the electrode pads 615 and 616 to form an electrode pair 637 and the electrode pads 616 and 611 to form an electrode pair 638 in step S430. In some embodiments, the control unit 130 can synchronously measure the EMG signals sEMG of the electrode pairs 631-638 through the signal-capturing module 120. In some other embodiments, the control unit 130 can sequentially scan and measure the muscle area 1201 where the electrode pairs 631-638 are located at in clockwise way through the signal-capturing module 120. In the rest embodiments, the control unit 130 can sequentially scan and measure the muscle area 1201 where the electrode pairs 631-638 are located at in counterclockwise way through the signal-capturing module 120.

FIG. 13 is an application situation diagram of the electrode patch module 110 of FIG. 12 according to an embodiment of the disclosure. In the application of FIG. 13, different electrode patch modules 110 (as shown by FIG. 12) are affixed on different zones to be tested 1301 and 1302. The black round spots in the zones to be tested 1301 and 1302 represent chronic pain spots. The user can affix the electrode patch modules 110 shown by FIG. 12 in any directions at consciously felt chronic pain places by the user (for example, the zones to be tested 1301 and 1302). The electrode pads 1, 2, 3, 4, 5 and 6 shown by FIG. 13 respectively and correspondingly represent the electrode pads 612, 611, 616, 615, 614 and 613 shown by FIG. 12. The signal-capturing module 120 in step S430 selects the electrode pads 1 and 2 to form a first electrode pair, the electrode pads 2 and 3 to form a second electrode pair, the electrode pads 3 and 4 to form a third electrode pair, the electrode pads 4 and 5 to form a fourth electrode pair, the electrode pads 5 and 6 to form a fifth electrode pair and the electrode pads 6 and 1 to form a sixth electrode pair. In the embodiment, the control unit 130 in step S440 can sequentially scan and measure the first, second, third, fourth, fifth and sixth electrode pairs in counterclockwise way through the signal-capturing module 120.

As shown by FIG. 13, in the zone to be tested 1301, the direction of the second electrode pair (i.e., the electrode pads 2 and 3) and the direction of the fourth electrode pair (i.e., the electrode pads 4 and 5) are parallel to the muscle trending direction, therefore, the EMG signals sEMG (or characteristic values) of the second and fourth electrode pairs are relatively greater than the signal of the adjacent electrode pair. In step S450, the control unit 130 can judge out the second and fourth electrode pairs are parallel to the fascicle direction according to the EMG signals sEMG (or characteristic values) of the electrode pairs. Then, the control unit 130 in step S450 can decide the chronic pain position through comparing the characteristic values of the second and fourth electrode pairs with each other. In the situation of FIG. 13, since the fourth electrode pair is closest to the chronic pain spot (black round spot in FIG. 13), the characteristic value of the fourth electrode pair should be greater than the characteristic value of the second electrode pair. According to the characteristic values of the electrode pairs, in step S450, the control unit 130 can judge out the chronic pain spot is located at the place of the fourth electrode pair. Analogy for the rest, the control unit 130 can judge out the chronic pain spot in the zone to be tested 1302 is located at the place of the sixth electrode pair (i.e., the electrode pads 6 and 1).

In summary, the embodiments of the disclosure use a chronic pain detection method of an electrode patch module and the apparatus thereof. The electrode patch module has at least three electrode pads. A plurality of electrode pairs are selected from the electrode pads. In some embodiments, a user during measuring physiological characteristics is not required to position the electrode pairs according to the muscle stria directions, instead, it is required to affix the electrode patch module at a zone to be tested only. By using the electrode pairs to measure the physiological characteristics of different muscle areas in the zone to be tested, the embodiment of the disclosure can reveal the chronic pain locations in the zone to be tested through the chronic pain detection method of the electrode patch module. The embodiments allow a common user finding out the chronic pain spots on the muscle to be tested without considering the direction of the electrode patch module through analyzing surface EMG signals. The user can affix the electrode patch module on the muscle to be tested without the aid of the professionals and is aware of the chronic pain locations on the muscle to be tested by means of the chronic pain detection method.

It will be apparent to those skilled in the art that various modifications and variations can be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. A chronic pain detection method by using electrode patch module, comprising:
providing an electrode patch module, wherein the electrode patch module contains at least three electrode pads, and a plurality of electrode pairs are selected from the electrode pads;
measuring electromyography signals of the electrode pairs to obtain at least one characteristic value; and
performing a comparison operation on the characteristic values to produce a judgment result.

2. The chronic pain detection method by using electrode patch module as claimed in claim 1, wherein the step of measuring electromyography signals of the electrode pairs comprises:
respectively measuring electromyography signals of different muscle areas through the electrode pairs; and
respectively calculating the characteristic values of the electromyography signals.

3. The chronic pain detection method by using electrode patch module as claimed in claim 2, wherein in the step of measuring electromyography signals through the electrode pairs, the electrode pairs synchronously measure the electromyography signals.

4. The chronic pain detection method by using electrode patch module as claimed in claim 2, wherein the step of obtaining the characteristic values of the electromyography signals comprises:
respectively calculating an average amplitude of the electromyography signals; and
respectively taking the average amplitudes as the characteristic values of the electromyography signals.

5. The chronic pain detection method by using electrode patch module as claimed in claim 1, wherein the comparison operation comprises:
defining a chronic pain index;
comparing the characteristic values with the chronic pain index;
if one value among the characteristic values is greater than or equal to the chronic pain index, judging out a muscle area corresponding to the value is chronic pain; and
if one value among the characteristic values is less than the chronic pain index, judging out a muscle area corresponding to the value is normal.

6. The chronic pain detection method by using electrode patch module as claimed in claim 5, wherein the step of defining a chronic pain index comprises:
comparing the characteristic values with each other to find out a minimal value among the characteristic values; and
setting N times of the minimal value among the characteristic values as the chronic pain index, wherein N is a positive real number.

7. The chronic pain detection method by using electrode patch module as claimed in claim 6, wherein N is equal to 3.

8. The chronic pain detection method by using electrode patch module as claimed in claim 1, wherein the electrode pairs respectively have a first electrode pad and a second electrode pad; the first electrode pad and the second electrode pad in a same electrode pair have an interval therebetween; and the intervals of the electrode pairs are equal to each other.

9. The chronic pain detection method by using electrode patch module as claimed in claim 1, wherein the electrode pairs respectively have a first electrode pad and a second electrode pad; an axis is defined between the first electrode pad and the second electrode pad in a same electrode pair; and the axes of the electrode pairs are parallel to each other.

10. A chronic pain detection apparatus, comprising:
an electrode patch module, which contains at least three electrode pads;
a signal-capturing module, coupled to the electrode patch module, which selects a plurality of electrode pairs in the electrode pads; and
a control unit, coupled to the signal-capturing module, which measures electromyography signals of the electrode pairs through the signal-capturing module to obtain at least one characteristic value, and performs a comparison operation on the characteristic values to produce a judgment result.

11. The chronic pain detection apparatus as claimed in claim 10, wherein the signal-capturing module measures electromyography signals of different muscle areas through the electrode pairs, and sends the electromyography signals of different muscle areas to the control unit; and the control unit respectively calculates the characteristic values of the electromyography signals.

12. The chronic pain detection apparatus as claimed in claim 11, wherein the signal-capturing module synchronously measures the electromyography signals through the electrode pairs.

13. The chronic pain detection apparatus as claimed in claim 11, wherein the control unit respectively calculates an average amplitude of the electromyography signals; and the control unit respectively takes the average amplitudes as the characteristic values of the electromyography signals.

14. The chronic pain detection apparatus as claimed in claim 10, wherein the comparison operation comprises:
defining a chronic pain index;
comparing the characteristic values with the chronic pain index;
if one value among the characteristic values is greater than or equal to the chronic pain index, judging out a muscle area corresponding to the value is chronic pain; and
if one value among the characteristic values is less than the chronic pain index, judging out a muscle area corresponding to the value is normal.

15. The chronic pain detection apparatus as claimed in claim 14, wherein defining a chronic pain index comprises:
comparing the characteristic values with each other to find out a minimal value among the characteristic values; and
setting N times of the minimal value among the characteristic values as the chronic pain index, wherein N is a positive real number.

16. The chronic pain detection apparatus as claimed in claim 15, wherein N is equal to 3.

17. The chronic pain detection apparatus as claimed in claim 10, wherein the electrode pairs respectively have a first electrode pad and a second electrode pad; the first electrode pad and the second electrode pad in a same electrode pair have an interval therebetween; and the intervals of the electrode pairs are equal to each other.

18. The chronic pain detection apparatus as claimed in claim 10, wherein the electrode pairs respectively have a first electrode pad and a second electrode pad; an axis is defined between the first electrode pad and the second electrode pad in a same electrode pair;
and the axes of the electrode pairs are parallel to each other.

19. An electrode patch module, containing:
at least three electrode pads, which form a plurality of electrode pairs, the electrode pairs respectively have a first electrode pad and a second electrode pad, the first electrode pad and the second electrode pad in a same electrode pair have an interval therebetween, and the intervals of the electrode pairs are equal to each other.

20. The electrode patch module as claimed in claim 19, wherein an axis is defined between the first electrode pad and the second electrode pad in a same electrode pair, and the axes of the electrode pairs are parallel to each other.

21. The electrode patch module as claimed in claim 19, wherein the electrode pads are disposed on a quasi-circle, and the quasi-circle comprises circle shape or oval shape.
